(19) United States
(12) Patent Application Publication
(10) Pub. No.: US 2009/0123956 A1
(43) Pub. Date: May 14, 2009

(54) Measurement of the oxidants-antioxidants balance in liquids

(75) Inventors: George Koliakos, Thessaloniki (GR); Daryoush Hamidi Alamdari, Thessaloniki (GR)

Correspondence Address: KOLIAKOS GEORGE, 27b Vryoulon Str., 551 32 Thessaloniki, Greece, THESSALONIKI, GR

(73) Assignee: Samsung Electronics Co., Ltd., Suwon-si (KR)

(21) Appl. No.: 12/285,426

(22) Filed: Oct. 3, 2008

(30) Foreign Application Priority Data
Apr. 6, 2006 (GR) 060100213

(51) Int. Cl. C12Q 1/26

(52) U.S. Cl. 435/25

(57) ABSTRACT

The present invention describes a chemical method that can determine the oxidant-antioxidant balance in biological samples and other materials. In the example of the application of the invention that is presented in the description of the invention section 3,3′,5,5′-Tetramethylbenzidine (TMB) and its cation that has a characteristic color are used as an oxidation-reduction target. However any other substance that can change its optical, fluorescence luminescence properties upon oxidation or reduction could be used in its place. The invention can be applied in any shape of vessel and on a stable matrix as a dipstick. The invention is based on two reactions one redox and one enzymatic that take place at the same time. In a redox reaction, e.g. TMB cation will be reduced by antioxidants; in the enzymatic reaction, intact TMB will be oxidized by peroxides. In the process of reduction, TMB cation will be decolourized; and in the process of oxidation, intact TMB will be converted to a colour cation. After a period of the time and adding HCl, the amount of TMB cation can be easily measured by spectrophotometry at 450 nm (reference wavelength 620 or 570 nm) both by macro- and micromethods (ELISA reader). The quantitative amount of TMB cation is representative of the oxidants-antioxidants balance in sample. This is achieved by comparing the optical absorbance of each sample with the absorbance of a series of standards that comprise the standard curve. The standard solutions can be constructed by mixing varying proportions (0-100%) of hydrogen peroxide (as a representative of the oxidants) with uric acid (as a representative of the antioxidants). However the admixture of any other oxidant-antioxidant may be used. The invention can be useful for the evaluation of the oxidant-antioxidant balance in biological samples (serum, plasma, urine etc.) especially for the evaluation of age related and metabolic disease such as diabetes. In addition the invention may be useful for the estimation of the success of antioxidant therapy.

### FIELD OF THE INVENTION

The present invention concerns a method of determining the oxidants-antioxidants balance of a sample by using a substance that changes its optical properties (such as light absorbance, fluorescence or luminescence) upon oxidation. According to the invention, the oxidants-antioxidants balance is determined in this way, in a redox reaction. The substance will be reduced by antioxidants and changes its optical properties; in the enzymatic reaction, the reduced substance will be oxidized by an enzymatic reaction and regains its initial optical properties; the final concentration of one form of the substance is measured and compared with a standard curve. The standard curve is constructed by mixing varying proportions (0-100%) of an oxidant such as hydrogen peroxide (as a representative of the oxidants) with an antioxidant such as uric acid (as a representative of the antioxidants). According to our best knowledge, this measurement of the oxidants-antioxidants balance is reported for the first time.

### BACKGROUND OF THE INVENTION

Reduction of a chemical is defined as a gain of electrons. Oxidation is defined as a loss of electrons. A reductant or a reducing agent is a substance that donates electrons and, thereby, causes another reactant to be reduced. An oxidant or an oxidizing agent is a substance that accepts electrons and causes another reactant to be oxidized. An oxidation is impossible without a reduction elsewhere in the system. Reductant and oxidant are chemical terms, whereas antioxidant and prooxidant have meaning in the context of a biological system.

In human body, reactive oxygen species (ROS, as pro-oxidants) such as the hydroxyl radical (OH.), the superoxide radical (O.₂), the nitric oxide radical (NO.) and the lipid peroxyl radical (LOO.) are derived either from normal essential metabolic processes or from external sources such as exposure to X-rays, ozone, cigarette smoking, air pollutants and industrial chemicals (Dean, R. T., et al, Biochem. J. (1997), 324:1-18).

ROS can readily react with and damage other molecules that in some cases, the body uses this to fight infections, or in other cases, the damage may be to the body's own molecules such as DNA, lipid, proteins and carbohydrates (Stadtman, E. R. Annu. Rev. Biochem. (1993), 62:797-821).

In body, there is always a balance between pro-oxidants and antioxidants and the antioxidants mop up ROS before they damage other essential molecules. The antioxidants were defined as enzymes or molecules that can retard or prevent the damaging effects of ROS in tissues. Clinical trials and epidemiological studies have established an inverse correlation between the intake of antioxidants and the occurrence of disease such as inflammation, cardiovascular disease, cancer, and aging-related disorders (Halliwell, B.; Gutteridge, J. Oxford Univ. Press, NY (1999)); (Willet, W. C., The Harvard Medical School Guide to Healthy Eating; Simon and Schuster: New York (2001)).

To date, various methods have been developed to measure the total antioxidant capacity (Cao G, Prior R L. Clin Chem. (1998), June; 44(6 Pt 1):1309-15); (Frankel E N, Meyer A S. J Sci Food Agr (2000), 80 (13): 1925-1941 October Review); (Prior R L, et al, J Agric Food Chem. (2005), May 18; 53(10):4290-302. Review); (Benzie I F, Strain J J. Anal Biochem. (1996), July 15; 239(1):70-6) and the total oxidants of a sample by a chemical way (FOX-1 assay, FOX-2 assay; Wolff S. P., Method Enzymol 1994, 233: 182-189; Sodergren E, et al, J Biochem Biophys Methods. 1998 Nov. 18; 37(3):137-46; Gay C A, Gebicki J M. Anal Biochem. 2003 Apr. 1; 315(1):29-35; Erel O., Clin Biochem. 2005 Oct. 5, Epub ahead of print) or by a enzymatic way (peroxide assay; FIG. 2; Franz T., et al; Anal. Biochem. 2003, May 15; 316(2):147-53), separately.

Also plasma (or serum) concentrations of different oxidants or different antioxidants can be measured in laboratories separately, but the measurements are time-consuming, labor-intensive, costly and they require complicated techniques. Because the measurement of different oxidants or different antioxidant molecules separately, are not practical and oxidants effects or antioxidant effects of them are additive, the total oxidant statue (TOS) and the total antioxidant response (TAR) of a sample are measured and these are named as total peroxide (TP) (Harma M, et al, Am J Obstet Gynecol 2005; 192(2):656-7; Yeni E, et al, 2005; 17(1): 19-22), serum oxidation activity (SOA) (Nakamura K, et al, Int J Tissue React 897; 9(4):307-16), reactive oxygen metabolise (ROM) (Ceylan E, et al, 2005; 72(2):156-9; Squitti R, et al, Eur J Clin Invest 2002; 32:51-9) or some other synonyms; and total antioxidant capacity, Miller N J, et al., Clin Sci (Lond). (1993), April; 84(4):407-12); (Re R, et al. Free Radic Biol Med. (1999), May; 26(9-10):1231-7), total antioxidant activity, total antioxidant power, total antioxidant status, or other synonyms (Prior R L, Cao G, Free Radic Biol Med. (1999); December; 27(11-12):1173-81. Review), respectively.

In the evaluation of oxidative stress status, all published methods involve separate measurement of the total oxidants status (TOS) and total antioxidants capacity (TAC); and until now, no method has been developed to measure the balance of oxidants and antioxidants at the same time with one step of experiment.

The performance of both measurements (TOS and TAC) is necessary for the right judgement of the oxidant-antioxidant balance; because the amount of TOS may be in normal range but TCA may be decreased or the amount of TOS may be increased but TCA may be in normal range or in some diseases just TAC is just increased.

The advantages of the present invention include the following: 1) the assay can be carried out in a single step for each sample, i.e. there is no series of separate reactions required to obtain the result; 2) the apparatus required is relatively simple and available, 3) the test permits several assays to be carried out simultaneously for a numbers of samples and 4) the test is cost effective.

### BRIEF DESCRIPTION OF THE INVENTION

The invention describes a method of determining the oxidants-antioxidants balance of a liquid and/or solution by using two different reactions in one single step; the first reaction is a chemical reaction where a chromogen e.g. the TMB cation or any other substance the chemical properties of which can be changed (light absorbance, fluorescence or luminescence) is reduced to a colourless compound (or a compound with different optical properties) by antioxidants during a redox reaction; another one is an enzymatic reaction where the substance e.g. TMB is oxidised into a colour cation (or a substance with the initial optical properties) by the enzyme peroxidase and peroxides like hydrogen peroxide (H₂O₂) or lipid hydroperoxide (ROOH); Therefore, after a period of time and adding HCl both reactions (chemical reaction and enzymatic reaction) are done, the remains of TMB cation can easily be measured by recording the absorbance signal at a specific wavelength (450 nm) with reference wavelength (620 nm or 570 nm); This absorbance is then compared with the absorbance given by a known concentration of a standard solutions that is made by mixing varying proportions (0-100%) of hydrogen peroxide (as a representative of the oxidants) with uric acid (as a representative of the antioxidants) at the same wavelength.

It is an object of the invention to provide a simple, efficient method and one step experiment for measuring oxidants-antioxidant balance of a sample.

The method can be used in liquid phase in any kind of vessel and also with the reagents adsorbed on a stable matrix as a dipstick.

In place of TMB other substances can be used such as a fluorescent substance e.g DCFDA or theachemiluminescent substance e.g. luminol.

Another important point of this invention is this: Hydrogen peroxide and uric acid are chosen as a representative of the oxidants and as a representative of the antioxidants, respectively; and the standard solutions is made by mixing varying proportions (0-100%) of hydrogen peroxide with uric acid, in the mixture, they have no interaction together and they do not neutralize the activity of each other. Therefore, the standard solutions can be made from these two reagents by mixing varying proportions.

In our pervious patent, “Measurement of the total antioxidant capacity in liquids and solutions by using of 3,3′, 5,5′-Tetramethylbenzidine (TMB)”, we described in detail the ability of antioxidants to reduce TMB cation which causes decolourization of TMB cation and hence results in a decrease in absorbance at 450 nm (reference wavelength 620 or 570 nm). By comparing the change in absorbance for an equivalent molar concentration of different reducing agents (i.e. antioxidants) with an equivalent molar concentration of uric acid, the relative activity of different reducing agents is calculated. The relative activity of vitamin C, Trolox, GSH, albumin and bilirubin in compare with uric acid were about 1, 1, 1, 0.6, 2 (FIG. 3 and FIG. 4).

Also GSH is an antioxidant whose concentration is low in human serum. However, the functional part of GSH as an antioxidant is the SH group, which is also present in non-protein antioxidants such as lipoic acid and some amino acids. Therefore, GSH was used here to represent the SH-group-containing non-protein compounds.

The assay used in the present invention is of use with many different types of samples. Thus it is applicable to biological fluids including mammalian serum or EDTA plasma, cerebrospinal fluid, synovial fluid or saliva. The assay is of further use in assaying the oxidants-antioxidants balance of tissues, cells or other materials such as foodstuffs and oils. Synovial fluid will provide useful information in the diagnosis and/or prognosis of arthritic or rheumatic disorders whereas CSF will provide possible implications of free radical induced nerve degeneration in the brain and spinal cord, and any possible role they may have in neural inflammation in diseased states or after injury.

In a further embodiment the invention can be used to determine the contribution of specific or particular classes of antioxidants known or suspected to be present in a sample, by comparing the oxidants-antioxidant balance as measured by the method of the invention before and after the specific or particular classes of antioxidants have been prescribed.

Similarly, the invention has application in monitoring the progress of patients suffering from the large number of diseases and infections that cause a change in the balance of pro-oxidants and antioxidants in body fluids.

A further application of the invention is in monitoring the effectiveness of antioxidants drugs. Thus, after administration of such a drug, the oxidants-antioxidant balance can be monitored to provide information on the rate of recovery.

### DETAILED EXAMPLE FOR THE APPLICATION OF THE INVENTION

Further features and advantages of the invention will be more readily apparent from the following description of a preferred embodiment of the method. According to the invention, the determination of the oxidants-antioxidants balance of a sample provides the following steps:

1. preparing a uric acid solution (6 mM) and a hydrogen peroxide solution (1 mM);
2. preparing substrate buffer (phosphate citrate buffer):
1.455 gr di-sodium hydrogen phosphate anhydrous (Na₂HPO₄₎, 1.91 gr citric acid anhydrous (C₆H₈O₇) is dissolved in 180 ml dH₂O, pH is adjusted at 5, the volume is adjusted up to 200 ml with dH₂O and is stored at 4° C.;
3. preparing hydrochloride acid solution (HCl:2N): 40 ml HCl 37% is added to 150 dH₂O and volume is adjusted up to 200 ml with dH₂O;
4. preparing ammonium persulfate (0.4%): 0.04 gr ammonium persulfate is dissolved in 10 ml distilled water, is dispensed in aliquots (80 μl) and stored at −20° C. for six months;
5. preparing the standard solutions by mixing varying proportions (0-100%) of hydrogen peroxide (as a representative of the oxidants) with uric acid (as a representative of the antioxidants);
6. preparing 3,3′,5,5′-Tetramethylbenzidine (TMB) buffer (freshly prepared before using):
One TMB.2HCl tablet (Sigma, contain 1 mg 3,3′,5,5′-Tetramethylbenzidine.2HCl) is dissolved in a Falcon tube (15 ml, in order to the solution be in dark, the falcon tube was wrapped by aluminum foil) that contains 10 ml substrate buffer (this solution is dividing in two part: 9 ml and 1 ml); 1 ml of this solution is transferred to in a eppendorf tube that was wrapped by aluminum foil. (unused TMB buffer can be stored for 4 days at 4° C.);
7. pouring a 10 μl quantity of each sample, standard and blank (distilled water) into the wells of a multiwell plate;
8. preparing TMB cation solution (freshly prepared before using): 10 μl of ammonium persulfate (0.4%) is added to the eppendorf tube that contains 1 ml of TMB solution and incubate at room temperature for 3 min;
9. adding 1.1 μl of peroxidase enzyme (horseradish peroxidase, the enzyme activity of stock solution is 5 KU/ml, Applichem: 230 U/mg, Order-NO. A3791,0005) into 9 ml TMB buffer, mixed well gently and used immediately (the enzyme activity of solution is 25 mU/ml);
10. preparing TMB solution by returning the TMB cation solution into 9 ml TMB buffer contains peroxidase enzyme and incubate it for 3 minute in room temperature and use it immediately;
11. adding 200 μl of TMB solution to each well and incubating it at room temperature in dark place for 12 minutes;
12. adding 100 μl hydrochloride acid solution (2N) to each well;
13. incubate the plate for 30-45 minutes in dark place;
14. carrying out a spectrophotometry of the samples at 450 nm with reference wavelength 620 or 570 nm by an ELISA reader;
15. providing a standard curve from the values relative to the standard samples by Microsoft Excel program and obtain a equation curve for it, balance in the standard curve, the absorbance (y axis) is plotted in versus the values of the oxidants-antioxidants balance (on x axis, the values of the oxidants-antioxidants balance are the percentage of antioxidant multiply by 6, its unit is HK that it is an arbitrary unit), the standard curve is shown in FIG. 1;
16. calculating the values of the unknown samples on the base of the values obtained from the above standard curve that the values are expressed as HK (an arbitrary unit): percentage of antioxidant multiply by 6 (HK unit);

### Example of Application 1

Using the above described method the oxidants-antioxidants balance is measured for 35 healthy Greece adults, 30 diabetic patients. The statistical analysis of two group of healthy individuals and diabetic show that there is a significant different between two group (P value is <0.001), healthy individuals have less value of oxidant-antioxidant balance than diabetic patients (table 1);

TABLE 1
the measured amount of the oxidants-antioxidants
balance for serum samples.

| | Control group n = 35 | | | Diabetic patients n = 30 | | |
|---|---|---|---|---|---|---|
| | range | mean | SD | range | mean | SD |
| Balance values | 49-105* | 75.8 | 15.3 | 86-141* | 110 | 17.1 |
| Age (years) | 26-53 | 39 | 18 | 28-56 | 43 | 15 |

*the values are expressed as HK (an arbitrary unit)

### Example for Application 2

Using the above described method serum of 8 diabetic patients were taken one day and one month after the consumption of vitamin C and E. The test was done for fresh and aged serum samples (one month at −80° C. or one week at −20° C.). The statistical analysis indicated that there is a significant different between before and after one day consumption of vitamin C and E (P value is <0.0033), and also between one day and one month (P value is <0.001), the results are presented in table 2;

TABLE 2
the measured amount of the oxidants-antioxidants balance for 8
diabetic patients before consumption of vitamin and after one day,
one month of consumption of vitamin C and E.

| | before | after one day | after one month |
|---|---|---|---|
| | mean* SD | mean* SD | mean* SD |
| balance | 118 ± 14 | 90.5 ± 14 | 75.7 ± 16 |
| value | | | |

*the values are expressed as HK (an arbitrary unit)

### DESCRIPTION OF THE ACCOMPANYING DRAWINGS

FIG. 1 shows oxidants-antioxidants balance in the standard curve, the absorbance (y axis) is plotted in versus the values of the oxidants-antioxidants balance (on x axis, the values of the oxidants-antioxidants balance are the percentage of antioxidant multiply by 6, its unit is HK that it is an arbitrary unit).

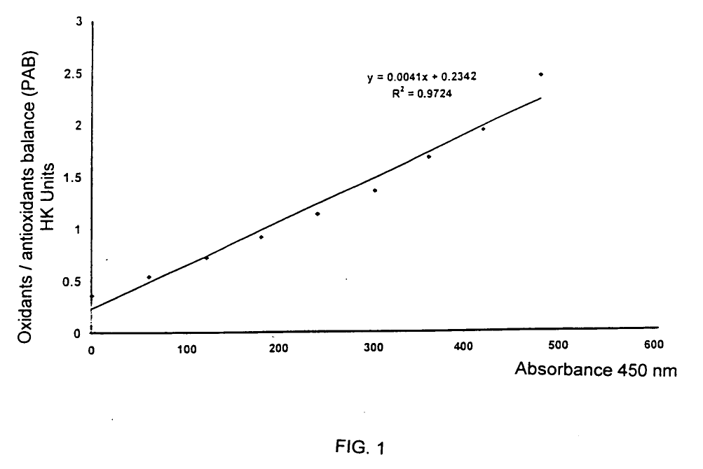

FIG. 2 shows standard curve for the determination of total peroxides by horseradish peroxidase enzyme and TMB substrate, the absorbance (y axis) is plotted in versus the values of hydrogen peroxide concentrations (on x axis), Franz T., et al, Anal. Biochem. 2003, May 15; 316(2):147-53.

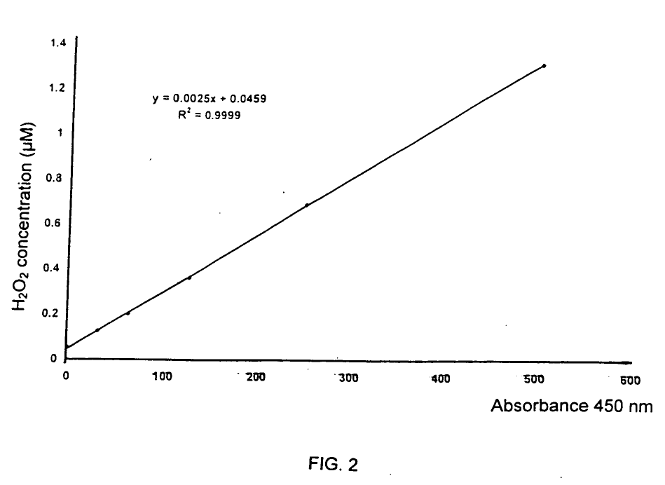

FIG. 3 shows the rate of increase for standards solutions (the subtraction of the blank absorbance from the standards solutions absorbance at 450 nm (reference wavelength 620 or 570 nm), uric acid (filled triangles), ascorbic acid (cross), trolox (filled circles), GSH (cross square) and bilirubin (open squares), all measured in parallel (Hamidi Alamdari D., Koliakos G., filled Patent NO 20050100503, 3 Oct. 2005, Greece).

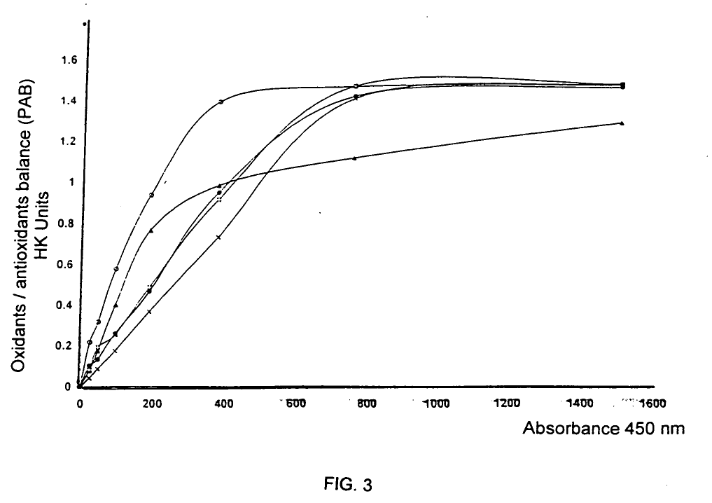

FIG. 4 shows the rate of increase for standard solutions of Albumin (the subtraction of the blank absorbance from the standard solutions absorbance at 450 nm (reference wavelength 620 or 570 nm) (Hamidi Alamdari D., Koliakos G., filled Patent NO 20050100503, 3 Oct. 2005, Greece).

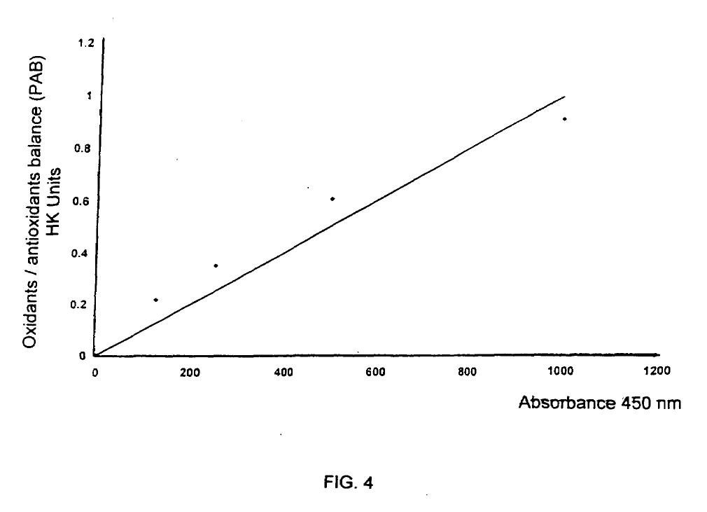

### REFERENCES CITED

### Patent

Hamidi Alamdari D., Koliakos G., “Measurement of the total antioxidant capacity in liquids and solutions by using of 3,3′,5,5′-Tetramethylbenzidine (TMB)”, filled Patent NO 20050100503, 3 Oct. 2005, Greece.

### OTHER REFERENCES

Benzie I F, Strain J J. The ferric reducing ability of plasma (FRAP) as a measure of “antioxidant power”: the FRAP assay. Anal Biochem. 1996 Jul. 15; 239(1):70-6.
Cao G, Prior R L. Comparison of different analytical methods for assessing total antioxidant capacity of human serum. Clin Chem. 1998 June; 44(6 Pt 1):1309-15.
Ceylan E, Gulsun A, Gencer M, Aksoy N. A new parameter in the detection of tuberculosis activity: reactive oxygen metabolites. Respiration 2005; 72(2):156-9.
Dean, R. T.; Fu, S.; Stoker, R.; Davies, M. J. Biochemistry and pathology of radical-mediated protein oxidation. Biochem. J. 324:1-18; 1997.
Erel O., A new automated colorimetric method for measuring total oxidant status. Clin Biochem. 2005 Oct. 5; [Epub ahead of print]
Frankel E N, Meyer A S. The problems of using one-dimensional methods to evaluate multifunctional food and biological antioxidants. J Sci Food Agr 80 (13): 1925-1941 October 2000. Review.
Franz T., Sirid G., Willibald W., Rudolf W., Dual method for the determination of peroxidase-activity and total peroxides—Iodide leads to a significant increase of peroxidase-activity in human sera. Anal. Biochem. 2003, May 15; 316(2):147-53.
Gay C, Collins J, Gebicki J M., Hydroperoxide assay with the ferric-xylenol orange complex. Anal Biochem. 1999 Sep. 10; 273(2):149-55.
Gay C A, Gebicki J M. Measurement of protein and lipid hydroperoxides in biological systems by the ferric-xylenol orange method. Anal Biochem. 2003 Apr. 1; 315(1):29-35.
Halliwell, B.; Gutteridge, J. Free radicals in biology and medicine. Oxford Univ. Press, NY; 1999.
Harma M, Harma M, Erel O. Oxidative stress in women with preeclampsia. Am J Obstet Gynecol 2005; 192(2):656-7.
Miller N J, Rice-Evans C, Davies M J, Gopinathan V, Milner A. A novel method for measuring antioxidant capacity and its application to monitoring the antioxidant status in premature neonates. Clin Sci (Lond). 1993 April; 84(4):407-12.
Nakamura K, Endo H, Kashiwazaki S. Serum oxidation activities and rheumatoid arthritis. Int J Tissue React 1897; 9(4):307-16.
Prior R L, Cao G. In vivo total antioxidant capacity: comparison of different analytical methods. Free Radic Biol Med. 1999 December; 27(11-12):1173-81. Review.
Prior R L, Wu X, Schaich K. Standardized methods for the determination of antioxidant capacity and phenolics in foods and dietary supplements. J Agric Food Chem. 2005 May 18; 53(10):4290-302. Review.
Re R, Pellegrini N, Proteggente A, Pannala A, Yang M, Rice-Evans. Antioxidant activity applying an improved ABTS radical cation decolorization assay. Free Radic Biol Med. 1999 May; 26(9-10):1231-7.
Sodergren E, Nourooz-Zadeh J, Berglund L, Vessby B., Re-evaluation of the ferrous oxidation in xylenol orange assay for the measurement of plasma lipid hydroperoxides. J Biochem Biophys Methods. 1998 Nov. 18; 37(3):137-46.
Squitti R, Rossini P M, Cassetta E, et al. D-Penicillamine reduces serum oxidative stress in Alzheimer's disease patients. Eur J Clin Invest 2002; 32:51-9.
Stadtman, E. R. Oxidation of free amino acids and amino acid residues in proteins by radiolysis and by metal-catalyzed reactions. Annu. Rev. Biochem. 62:797-821; 1993.
Willet, W. C. Eat, Drink, and be Health—The Harvard Medical School Guide to Healthy Eating; Simon and Schuster: New York, 2001.
Wolff S. P., Ferrous ion oxidation in presence of ferric ion indicator xylenol orange for measurement of hydroperoxides., Method Enzymol 1994, 233: 182-189.
Yeni E, Gulum M, Selek S, et al. Comparison of oxidative/antioxidative status of penil corpus cavernosum blood and peripheral venous blood. Int J Impot Res 2005; 17(1):19-22.

1. A method of measurement of the balance of oxidants and antioxidants (PAB) in liquids, characterized in that it comprises in combination the steps of:
setting up a standard curve illustrating the variation of the balance of oxidants and antioxidants (PAB) in reference samples comprising varying proportions of hydrogen peroxide as a representative oxidant and of uric acid as a representative antioxidant in terms of the photometric absorbances exhibited thereby;
conducting a single, spatially non-separated assay of a sample of the liquid in which the balance of oxidants and antioxidants (PAB) is to be measured with a target substance that exhibits change in its optical properties upon oxidation or reduction comprising an oxy-reductive reaction and an enzymatic reaction, said reactions being carried out simultaneously for a predetermined period of time;
adding a predetermined HCL solution in said sample of the liquid in which the balance of oxidants and antioxidants (PAB) is to be measured with the target substance that exhibits change in its optical properties upon oxidation or reduction and measuring the value of the photometric absorbance exhibited by said sample at the end of said predetermined period of time, and
comparing said value of the photometric absorbance exhibited by said sample at the end of said predetermined period of time with the photometric absorbances exhibited by said reference samples comprising varying proportions of hydrogen peroxide as a representative oxidant and of uric acid as a representative antioxidant, henceforth determining the balance of oxidants and antioxidants (PAB) in said sample of the liquid in which the balance of oxidants and antioxidants (PAB) is to be measured.

2. A method of measurement of the balance of oxidants and antioxidants (PAB) in liquids, as claimed in claim 1, characterized in that said target substance that exhibits change in its optical properties upon oxidation or reduction is 3,3′,5,5′-Tetramethylbenzidine (TMB), said substance exhibiting a change of colour upon oxidation or reduction, wherein said enzymatic reaction comprises oxidation of the chromogen TMB to a color cation by peroxides and said oxy-reductive reaction comprises reduction of said TMB cation to a colorless compound by antioxidants.

3. A method of measurement of the balance of oxidants and antioxidants (PAB) in liquids, as claimed in claim 1, characterized in that said target substance that exhibits change in its optical properties upon oxidation or reduction is selected from the group consisting of 3,3′,5,5′-Tetramethylbenzidine (TMB) or (OPD), or 2,2′-azinobis-3-ethylbenzo-thiazoline-6-sulfonic acid (ABTS).

4. A method of measurement of the balance of oxidants and antioxidants (PAB) in liquids, as claimed in claim 1, characterized in that said target substance that exhibits change in its optical properties upon oxidation or reduction is DCFDA or other substance that exhibits a change in its fluorescence properties upon oxidation or reduction.

5. A method of measurement of the balance of oxidants and antioxidants (PAB) in liquids, as claimed in claim 1, characterized in that said target substance that exhibits change in its optical properties upon oxidation or reduction is luminol or other substance that exhibits a change in its luminescence properties upon oxidation or reduction.

6. A method of measurement of the balance of oxidants and antioxidants (PAB) in liquids, as claimed in claims 1-5, characterized in that said single, spatially non-separated assay of a sample of the liquid in which the balance of oxidants and antioxidants (PAB) is to be measured with a target substance that exhibits change in its optical properties upon oxidation or reduction comprising an oxy-reductive reaction and a simultaneous enzymatic reaction is being performed in a vessel of any form whatsoever.

7. A method of measurement of the balance of oxidants and antioxidants (PAB) in liquids, as claimed in claims 1-5, characterized in that said single, spatially non-separated assay of a sample of the liquid in which the balance of oxidants and antioxidants (PAB) is to be measured with a target substance that exhibits change in its optical properties upon oxidation or reduction comprising an oxy-reductive reaction and a simultaneous enzymatic reaction is being performed with the reagents absorbed on a stable matrix as a dipstick.
